(19) United States
(12) Patent Application Publication
(10) Pub. No.: US 2021/0330358 A1
(43) Pub. Date: Oct. 28, 2021

(54) GRADUALLY EXPANDING LIMB RECONSTRUCTION SYSTEMS

(71) Applicant: Indius Medical Technologies Private Limited, Pune (IN)

(72) Inventors: Aditya INGALHALIKAR, Pune (IN); Sagar SATHAYE, Pune (IN); Manali KUNTE, Pune (IN)

(73) Assignee: Indius Medical Technologies Private Limited, Pune (IN)

(21) Appl. No.: 17/240,466

(22) Filed: Apr. 26, 2021

(30) Foreign Application Priority Data
Apr. 27, 2020 (IN) 202021017910

(51) Int. Cl. A61B 17/66; A61B 17/88

(52) U.S. Cl. CPC A61B 17/66; A61B 2017/561; A61B 17/8805

(57) ABSTRACT

The present disclosure relates to a gradually expanding limb reconstruction system (100) comprising at least one fixator mechanism (100a) and at least one distractor mechanism (100b) wherein distractor mechanism is a self-driven growing rod system (500) comprising at least one cylindrical static rod (502) with an internal bore; at least one piston rod (506), coaxially coupled with said static rod (502) and configured to distract longitudinally out of the static rod (502); at least one fluid receptacle component (508) in the internal bore of the static rod (502); at least one fluid source (510) configured to hold at least one sterile biocompatible fluid at a pre-determined pressure; at least one pressure and flow control unit (512) configured to release metered doses of the sterile biocompatible fluid from the fluid source (510) to the fluid receptacle component (508) via at least one fluid transfer port (514).

The present application takes priority from the previously filed Indian Provisional application number 202021017910 titled “GRADUALLY EXPANDING LIMB RECONSTRUCTION SYSTEMS” dated 27th of Apr., 2020.

### FIELD

The present disclosure relates to hydraulics-based limb reconstruction systems.

### BACKGROUND

Limb reconstruction is the phenomenon of reinstating a limb to its normal or a desirable physical structure by means of a surgical procedure. Limb reconstruction may be recommended after instances such as non-healing fractures, post-traumatic deformities, bone infection, congenital paediatric deformities, shortening of limb, bone tumours, osteomyelitis (bone infection) and the like.

During the reconstruction procedure, a break is created surgically in the bone to be reconstructed and the bone segments are distracted (pulled apart) very slowly over a pre-determined period of time. As a virtue of the natural healing tendency of the human bones, a callus that is initially formed at the break, continues to get formed until the distraction is complete and the gap is filled. In this process, the combined action of a fixator mechanism and a distractor mechanism bring about the desired reconstruction. The fixator mechanism provides a supporting framework to the bone segments resulting from the surgical break in the bone to be reconstructed and the distractor mechanism, inherent to the fixator mechanism or a separate setup undergoes gradual distraction, manipulates the position of the fixator mechanism and consequently the bone segments to bring about the reconstruction. Some of the reconstruction systems described in the prior art are (a) the fixator mechanism and the distractor mechanism outside the limb or (b) the fixator mechanism inside the limb but the distractor mechanism outside the limb or (c) the fixator mechanism and the distractor mechanism both inside the limb.

In a majority of the reconstruction procedures mentioned herein above, the distractor mechanism facilitates a stepped distraction of the break created in the bone. Distraction is effected at a rate of 3-4 lengthenings per day and may take around 2 months to complete. Furthermore, each time the distraction has to be effected by direct manual intervention of a medical practitioner or the patient himself. Even further, since the distraction has to be manually calibrated and executed every time, even a slight human error may result in undesirable results.

The inventors of the present disclosure provide a gradually expanding system for limb reconstruction that mitigates the afore-mentioned drawbacks.

### OBJECTS

It is an object of the present disclosure to provide a gradually expanding limb reconstruction system.

It is another object of the present disclosure to provide a gradually expanding limb reconstruction system which is automated.

It is yet another object of the present disclosure to provide gradually expanding limb reconstruction system wherein the distraction is gradual and smooth.

It is still another object of the present disclosure to provide a gradually expanding limb reconstruction system which is versatile.

It is yet another object of the present disclosure to provide gradually expanding limb reconstruction system which is medical practitioner-friendly.

### SUMMARY

The present disclosure provides a gradually expanding limb reconstruction system (100) comprising at least one fixator mechanism (100a) and at least one distractor mechanism (100b) wherein distractor mechanism (100b) is a self-driven growing rod system (500) comprising at least one cylindrical static rod (502) with an internal bore; at least one piston rod (506), coaxially coupled with said static rod (502) and configured to distract longitudinally out of the static rod (502); at least one fluid receptacle component (508) in the internal bore of the static rod (502); at least one fluid source (510) configured to hold at least one sterile biocompatible fluid at a pre-determined pressure; at least one pressure and flow control unit (512) selected from the group consisting of solenoid valve, aperture controlling valve and flow controlling valve configured to release metered doses of the sterile biocompatible fluid from the fluid source (510) to the fluid receptacle component (508) via at least one fluid transfer port (514).

### BRIEF DESCRIPTION OF ACCOMPANYING DRAWINGS

The present disclosure is illustrated in the accompanying non-limiting drawings, throughout which, reference letters indicate corresponding parts in the various figures.

FIG. 1 illustrates an embodiment of the gradually expanding limb reconstruction system (100) of present disclosure as an external multi-planar hexapod frame;

FIG. 2 illustrates an embodiment of the gradually expanding limb reconstruction system (100) of present disclosure as an internal fixator;

FIG. 3a illustrates an embodiment of gradually expanding limb reconstruction system (100) of present disclosure as external fixator with intramedullary nail for lengthening over nail (LON);

FIG. 3b illustrates an embodiment of gradually expanding limb reconstruction system (100) of present disclosure as external fixator without intramedullary nail for lengthening over nail (LON);

FIG. 4a illustrates an embodiment of the self-driven growing rod system (500) of present disclosure with an integrated fluid source (510);

FIG. 4b illustrates an embodiment of the self-driven growing rod system (500) of present disclosure with detachable fluid source (510);

FIG. 5a illustrates an embodiment of the self-driven growing rod system (500) of present disclosure with the flexible, inflatable, collapsible element (516) and a cap (504);

FIG. 5b illustrates an embodiment of the self-driven growing rod system (500) of present disclosure with the flexible, inflatable, collapsible element (516) and without a cap (504);

FIG. 6 illustrates an embodiment of the self-driven growing rod system (500) of present disclosure with the dynamic seal (518) and the cap (504).

FIG. 7 illustrates an embodiment of the self-driven growing rod system (500) of present disclosure with a jacket (520);

FIG. 8a illustrates an embodiment of the self-driven growing rod system (500) of present disclosure with fluid source in-line with the system;

FIG. 8b illustrates an embodiment of the self-driven growing rod system (500) of present disclosure with fluid source at an angle with the system.

### DESCRIPTION

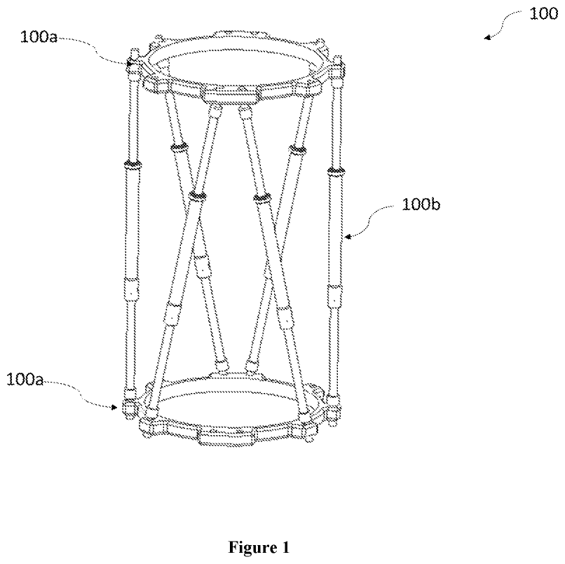

The present disclosure relates to a gradually expanding limb reconstruction system (100), as illustrated in FIGS. 1 to 3, comprising at least one fixator mechanism (100a) and at least one distractor mechanism (100b). The fixator mechanism (100a) provides a supporting framework to the bone segments resulting from the surgical break in the bone to be reconstructed and the distractor mechanism (100b) undergoes gradual distraction and manipulates the position of the fixator mechanism (100a) and consequently the bone segments to bring about the reconstruction.

The fixator mechanism (100a) of the present disclosure is at least one selected from the group consisting of screw(s), wire(s), hook(s), band(s), connector(s), plate(s), staple(s), nail(s) or any other fixation mechanism typically used in orthopedic surgery.

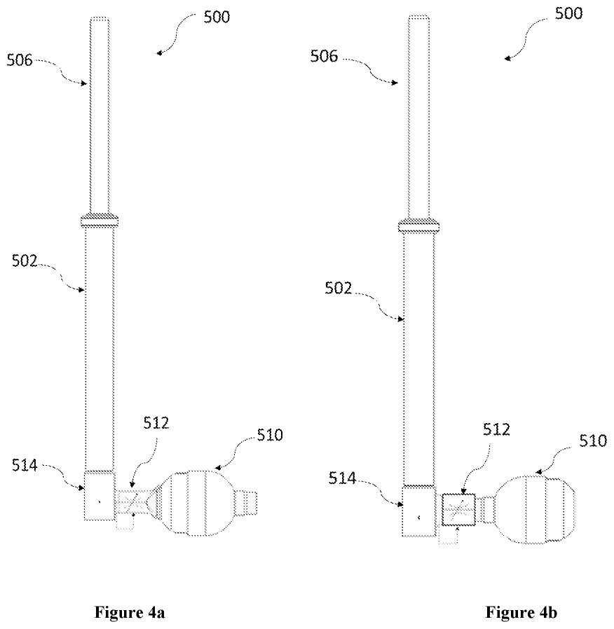

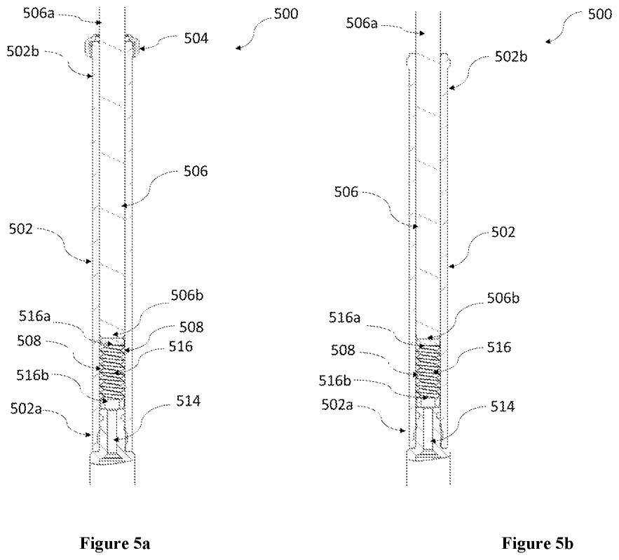

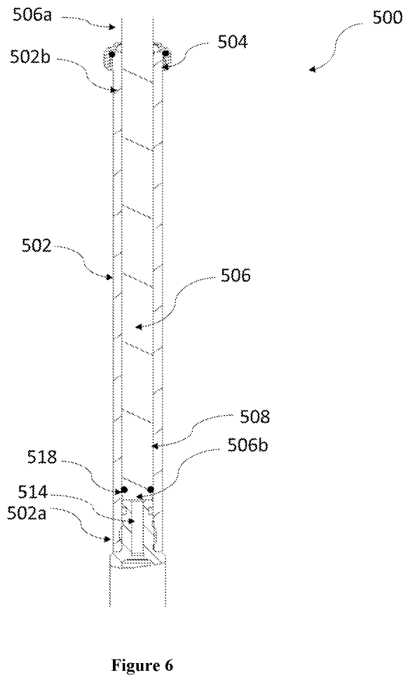

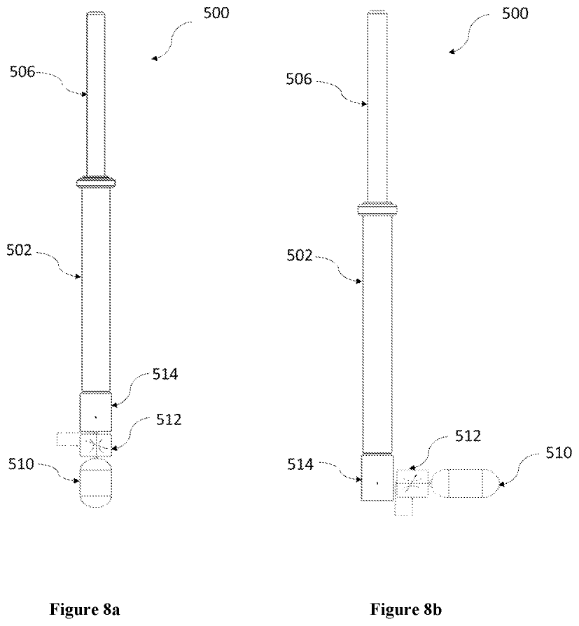

The distractor mechanism (100b) is a self-driven growing rod system (500), which is a characterizing feature of the present system (100). The self-driven growing rod system (500), illustrated in detail in FIGS. 4 to 8, comprises at least one component selected from the group consisting of at least one cylindrical static rod (502), at least one piston rod (506), at least one fluid receptacle component (508), at least one fluid source (510), at least one pressure and flow control unit (512) and at least one fluid transfer port (514).

The cylindrical static rod (502) is hollow and assumes the form of a cylinder with an internal bore and comprises a first static end (502a) and a second static end (502b). The first static end (502a) is affixed to the fixator mechanism (100a). The cylindrical static rod (502), functioning as the outer shell, hosts and provides support to all components housed therein.

The piston rod (506) of the present system (100) is coaxially coupled with the cylindrical static rod (502) and is configured to distract longitudinally out of the static rod (502) to facilitate its function of distraction. The first piston end (506a) of the piston rod (506) is configured for fixation on to the fixator mechanism (100a) and the second piston end (506b) of the piston rod (506b) is disposed within the internal bore of the static rod.

The fluid receptacle component (508) of the present system (100) can be divided into two embodiments. In one embodiment, the fluid receptacle component (508) is the area inside the inner bore of the static rod (502), being characterized by the presence of at least one cap (504) at the second static end (502b) of the static rod (502) and at least one dynamic seal (518) at the point of contact of the second piston end (506b) of the piston rod (506) and the internal bore of the static rod (502) to prevent leakage of the sterile biocompatible fluid contained therein, to the external environment.

In another embodiment, the fluid receptacle component (508) is the area inside the inner bore of the static rod, being characterized by the presence of at least one flexible, inflatable, collapsible element (516) affixed to the second piston end (506b) of the piston rod (506) at a first end (516a) and connected to the fluid transfer port (514) at a second end (516b) and optionally, at least one cap (504) at the second static end (502b) of the static rod (502). The flexible, inflatable, collapsible element (516) is at least one selected from the group consisting of metal bellows, polymeric bellows, polymeric balloon and polymeric tube. The dynamic seal (518) is replaced by the flexible, inflatable, collapsible element to avoid the drawbacks associated with using dynamic seals.

The fluid source (510) is configured to hold at least one sterile biocompatible fluid selected from the group consisting of water, deionized water, saline solution and gas at a pre-determined pressure. The gas is at least one selected from the group consisting of carbon dioxide, argon and nitrogen. The sterile biocompatible fluid is present in a pre-determined pressure ranging from 2 to 100 bar. The fluid source (510) is at least one selected from the group consisting of a permanently integrated pressure compensating device (illustrated in FIG. 4a) and a detachable pressure compensating device (illustrated in FIG. 4b). In one embodiment, the permanently integrated pressure compensating device is an accumulator. In said embodiment, a minimally invasive surgery may be performed if the pressure inside the system needs to be increased. In one embodiment, the permanently integrated pressure compensating device is in-line with the system (100). In another embodiment, the permanently integrated pressure compensating device is at an angle with the system (100). The detachable pressure compensating device is at least one selected from the group consisting of accumulators, fluid pumps, manual syringes, conventional vascular access ports or devices powered by electric motors or air compressors. In the embodiment where the fluid source (510) is detachable, optionally, at least one non-return valve (NRV) is provided at the fluid transfer port (514) to ensure that the sterile biocompatible fluid is contained within the system and doesn't leak out.

The pressure and flow control unit (512) is a characterizing feature of the self-driven growing rod system (500) used in the present limb reconstruction system. The pressure and flow control unit (512) is calibrated to release metered doses of the sterile biocompatible fluid from the fluid source (510) to the fluid receptacle component (508) via the fluid transfer port (514). Typically, the pressure and flow control unit (512) releases the sterile biocompatible fluid in an amount ranging from 5-2000 mm³. Depending on the end application, the amount of release of sterile biocompatible fluid is calibrated. The pressure and flow control unit (512) is at least one selected from the group consisting of solenoid valve, aperture controlling valve and flow controlling valve. The sterile biocompatible fluid having a constant, pre-determined pressure exiting the pressure and flow control unit (512) at a metered rate, enters into and fills the fluid receptacle component (508), applying force onto the piston rod (506), thereby causing the piston rod (506) to slide out of the cylindrical static rod (502) and effect distraction. Thus, the necessity of any direct manual intervention to cause distraction is obviated.

The fluid transfer port (514) of the present system (100) is configured to transfer the sterile biocompatible fluid from the pressure and flow control unit (512) to the fluid receptacle component (508).

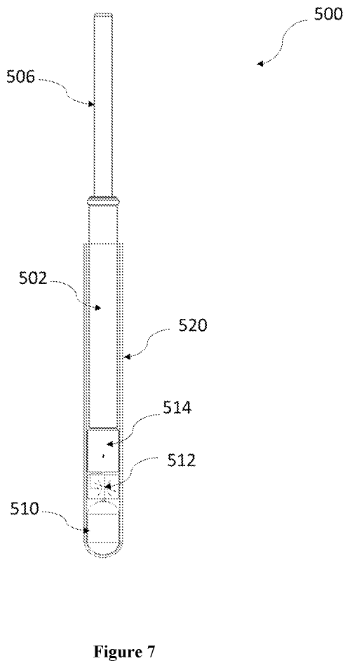

In the embodiment where the fluid source (510) is a permanently integrated pressure compensating device, at least one jacket unit (520) is optionally present to hold together the fluid source (510) and the other major components of the mechanism as illustrated in FIG. 7.

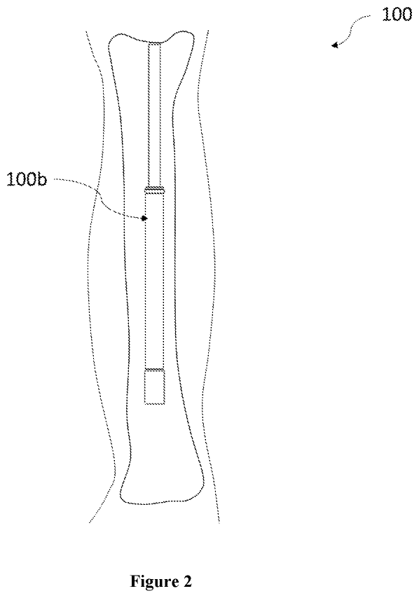

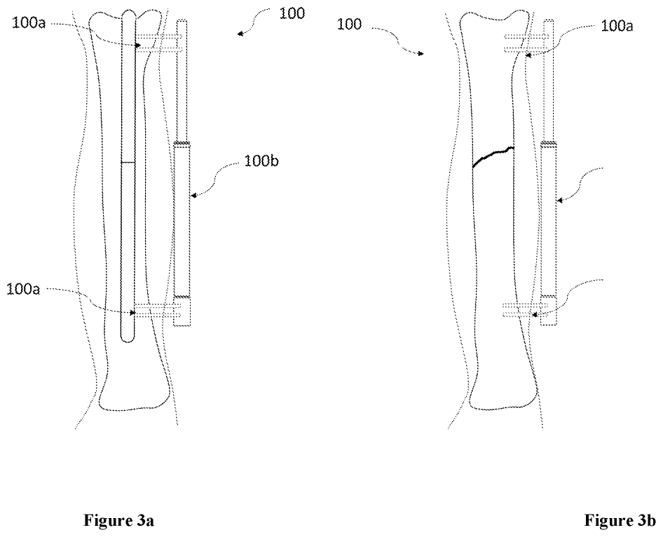

As a virtue of using a hydraulics-based self-driven growing rod system as the distractor mechanism (100b), the distraction caused is gradual and not stepped, as seen in the systems of the prior art. Further, as the distractor mechanism (100b) is self-driven (devoid of direct manual intervention), severe drawbacks associated with manual errors and repeated invasive surgeries are also obviated. Even further, the present system (100) provides for flexibility of approach and is adapted to work with a majority of the constructs known in the prior art such as external multi-planar hexapod frames (FIG. 1), internal fixators (FIG. 2), uniaxial external fixators with intramedullary nail for lengthening over nail (LON) (FIG. 3a) and uniaxial external fixators without intramedullary nail for lengthening over nail (LON) (FIG. 3b), making it extremely versatile. The hexapod arrangement (FIG. 1) may be used where both de-rotation and distraction is required. The struts of the arrangement can be differentially lengthened to facilitate de-rotation. Since the elongation mechanism is outside the limb in both multi-planar and uniplanar external constructs, all variations of the system in consideration can be used. A total of 10 variable constructs are possible with the present system (100) as represented herein below:

| | Variation | | | |
|---|---|---|---|---|
| | Detachable | Detachable | Integrated | Integrated |
| | Fluid Source | Fluid Source | Fluid Source | Fluid Source |
| | with Dynamic | without | with Dynamic | without |
| Construct | Seal | Dynamic Seal | Seal | Dynamic Seal |
| External Multi-planar | ✓ | ✓ | ✓ | ✓ |
| Hexapod Frame | | | | |
| Axial External | ✓ | ✓ | ✓ | ✓ |
| Fixator With and | | | | |
| Without | | | | |
| Intramedullary Nail | | | | |
| for Lengthening Over | | | | |
| Nail (LON) | | | | |
| Internal Fixator | X | X | ✓ | ✓ |

The afore-stated components of the system (100) of the present disclosure are manufactured from biocompatible materials. Further, the components of the system (100) of the present disclosure are manufactured from at least one material selected from the group consisting metal(s), metal alloys and polymers. For the purpose of the present disclosure, the term metal is at least one selected from the group consisting titanium, cobalt-chromium-molybdenum, and stainless steel or any other metal or metal alloy suitable from biocompatibility and strength perspective. For the purpose of the present disclosure, the term polymers is at least one selected from the group consisting high density polyethylene (HDPE), polyurethane, urethane, polycarbonate urethane, ultra-high molecular weight polyethylene (UHMWPE), polyethylene terephthalate (PET), polyether ether ketone (PEEK) and silicone or any other polymer suitable from biocompatibility and strength perspective. All the components of the system (100) of the present disclosure may be fabricated separately and attached together using conventional manufacturing techniques. The dimensions of the present system (100) may vary depending on factors such as age, weight, height, sex and the extent of reconstruction required by the patient.

The embodiments described herein above are non-limiting. The foregoing descriptive matter is to be interpreted merely as an illustration of the concept of the present disclosure and it is in no way to be construed as a limitation. Description of terminologies, concepts and processes known to persons acquainted with technology has been avoided to preclude beclouding of the afore-stated embodiments.

### Technical Advantages and Economic Significance

The technical advantages and economic significance of the gradually expanding limb reconstruction system (100) of the present disclosure are presented herein after:
Compatible with a majority of limb reconstruction constructs;
Distraction is automated;
Distraction is gradual and smooth;
Avoids errors associated with manual intervention.

We claim:

1. A gradually expanding limb reconstruction system (100) comprising:
a. at least one fixator mechanism (100a); and
b. at least one distractor mechanism (100b);
said system (100) being characterized in that said distractor mechanism (100b) is a self-driven growing rod system (500) comprising:
i. at least one cylindrical static rod (502) with an internal bore comprising a first static end (502a) and a second static end (502b);
ii. at least one piston rod (506), coaxially coupled with said static rod (502) and comprising a first piston end (506a) and a second piston end (506b); wherein said second piston end (506b) is disposed within the internal bore of said static rod (502) and configured to distract longitudinally out of the static rod (502);
iii. at least one fluid receptacle component (508);
iv. at least one fluid source (510) configured to hold at least one sterile biocompatible fluid at a pre-determined pressure; and
v. at least one pressure and flow control unit (512) configured to release metered doses of the sterile biocompatible fluid from the fluid source (510) to the fluid receptacle component (508) via at least one fluid transfer port (514).

2. The system (100) as claimed in claim 1, wherein said fixator mechanism (100a) is at least one selected from the group consisting of screw(s), wire(s), hook(s), band(s), connector(s), plate(s), staple(s) and nail(s).

3. The system (100) as claimed in claim 1, wherein said fluid receptacle component (508) is the area inside the inner bore of the static rod (502), being characterized by the presence of at least one flexible, inflatable, collapsible element (516) selected from the group consisting of metal bellows, polymeric bellows, polymeric balloon, polymeric tube, affixed to said second piston end (506b) of said piston rod (506) at a first end (516a) and connected to said fluid transfer port (514) at a second end (516b) and optionally, at least one cap (504) at said second static end (502b) of said static rod (502).

4. The system (100) as claimed in claim 1, wherein said fluid receptacle component (508) is the area inside the inner bore of the static rod (502), being characterized by the presence of at least one dynamic seal (518) at the point of contact of said second piston end (506b) of said piston rod (506) and the internal bore of said static rod (502) and at least one cap (504) at said second static end (502b) of said static rod (502).

5. The system (100) as claimed in claim 1, wherein said fluid source (510) is at least one selected from the group consisting of a permanently integrated pressure compensating device and a detachable pressure compensating device.

6. The system (100) as claimed in claim 1, wherein said sterile biocompatible fluid is at least one selected from the group consisting of water, deionized water, saline solution and gas, wherein said gas is at least one selected from the group consisting of carbon dioxide, argon and nitrogen.

7. The system (100) as claimed in claim 1, wherein said pressure and flow control unit (512) is at least one selected from the group consisting of solenoid valve, aperture controlling valve and flow controlling valve.

8. The system (100) as claimed in claim 1, wherein said fluid transfer port (514) is configured to transfer the sterile biocompatible fluid from the pressure and flow control unit (512) to the fluid receptacle component (508).

9. The system (100) as claimed in claim 1, further comprises at least one jacket (520) component to hold together the components of the present system.

10. The system (100) as claimed in claim 1, wherein the components are manufactured from biocompatible materials.
